(19) United States
(12) Patent Application Publication
(10) Pub. No.: US 2022/0229488 A1
(43) Pub. Date: Jul. 21, 2022

(54) Data Processing Device Executing Operation Based on User's Emotion

(71) Applicant: Semiconductor Energy Laboratory Co., Ltd., Kanagawa-ken (JP)

(72) Inventors: Kengo AKIMOTO, Isehara, Kanagawa (JP); Teppei OGUNI, Atsugi, Kanagawa (JP); Tatsuya OKANO, Isehara, Kanagawa (JP)

(73) Assignee: Semiconductor Energy Laboratory Co., Ltd., Kanagawa-ken (JP)

(21) Appl. No.: 17/617,107

(22) PCT Filed: Jun. 2, 2020
(86) PCT No.: PCT/IB2020/055189

§ 371 Date: Dec. 7, 2021

(30) Foreign Application Priority Data
Jun. 14, 2019 (JP) 2019-110985

(51) Int. Cl. G06F 3/01; G06V 40/16; B60W 40/08; G06T 7/00

(52) U.S. Cl. CPC G06F 3/011; G06V 40/169; B60W 40/08; G06T 2207/20081; B60W 2040/0872; G06T 2207/30201; G06T 2207/20084; G06T 7/00

(57) ABSTRACT

Provided are a data processing device and a data processing method that allow selecting and performing of an appropriate operation according to user's emotions. A user's face is detected and features of the user's face are extracted from data on the detected face. The user's emotions are estimated from the extracted facial features, and data based on the estimated user's emotions is generated. Whether the generated data is transmitted to an external device is determined on the basis of positional data of the user and the external device including a data reception unit, which is included in a radio wave transmitted from the Global Positioning System. When it is determined that the generated data is transmitted, it is transmitted to the external device. The external device that has received the data selects and executes operation based on the received data.

### TECHNICAL FIELD

One embodiment of the present invention relates to a data processing device and a data processing method.

Note that one embodiment of the present invention is not limited to the above technical field. Examples of the technical field of one embodiment of the present invention disclosed in this specification and the like include a semiconductor device, a display device, a light-emitting device, a power storage device, a memory device, an electronic device, a lighting device, an input device, an input/output device, a driving method thereof, and a manufacturing method thereof. In this specification and the like, a semiconductor device generally means a device that can function by utilizing semiconductor characteristics.

### BACKGROUND ART

Techniques of recognizing facial expressions from captured images of faces are known. Facial expression recognition is applied, for example, to a technique by which a digital camera or the like captures images automatically at the moment when a person smiles or when the person stares into the camera.

As a technique of facial expression recognition, for example, Patent Document 1 discloses a technique in which facial feature points are detected and facial expressions are recognized with high accuracy on the basis of the feature points.

### REFERENCE

### [Patent Document]

### [Patent Document 1] Japanese Published Patent Application No. 2007-087346

### SUMMARY OF THE INVENTION

### Problems to be Solved by the Invention

Actions (movement, behavior) of the human depend not a little on emotions of the moment, and slightly vary in many cases even when he or she has an intention of carrying out the same action. In driving a car, for example, a driver with a calm mind can drive a car safely; however, if the driver feels intense anger to be lost him/herself, driving maneuvers such as steering wheel operation and gas pedal depression might become violent. A driver who is in deep sadness might have impaired judgment, causing a slight delay in the timing of a variety of driving maneuvers. If a driver encounters some unexpected situation in driving and has a feeling of great surprise, he or she might lose the ability to make calm judgments and might make unthinkable mistakes, such as mistaking the gas pedal for the brake.

In operation such as driving a car, a slightest error in operation can directly lead to loss of life. Therefore, the driver must always have a high level of concentration and calm judgment, as well as the ability to perform stable driving operation that is not influenced by emotions of the moment. However, there are many cases in which the driver is not aware of the driving operation caused by the difference in the driver's emotions as described above, even if it is obvious to others, and it is difficult for the driver to carry out stable driving operation only by his or her own will.

Note that driving a car is given as an example here; the operation that could be influenced by human emotions is not limited thereto. For example, the operation of heavy machinery and the operation of equipment in a factory production line are also actions that could be influenced by human emotions.

An object of one embodiment of the present invention is to provide a device or a method for preventing abnormal operation due to human emotions. Another object of one embodiment of the present invention is to provide a device or a method that allows selecting and performing of an appropriate operation according to human emotions.

Another object of one embodiment of the present invention is to provide a novel data processing device. Another object of one embodiment of the present invention is to provide a novel data processing method.

Note that the description of these objects does not preclude the existence of other objects. One embodiment of the present invention does not have to achieve all these objects. Note that objects other than these can be derived from the description of the specification, the drawings, the claims, and the like.

### Means for Solving the Problems

One embodiment of the present invention is a data processing device including a subject detection unit detecting the face of a user; a feature extraction unit extracting a feature of the face; an emotion estimation unit estimating an emotion of the user from the feature; a data generation unit generating first data based on the estimated emotion; a sensor unit receiving a radio wave from the Global Positioning System; a data processing unit receiving the first data and second data included in the radio wave and transmitted from the sensor unit and generating third data based on the first data and the second data; and a data transmission unit transmitting the third data.

In the above, the third data is preferably transmitted to an external device including a data reception unit, the position of which is specified by the Global Positioning System.

In the above, the feature preferably includes at least one of the eye shape, the eyebrow shape, the mouth shape, the gaze, and the complexion of the user.

In the above, the feature is preferably extracted by inference using a neural network.

In the above, the emotion preferably includes at least one of anger, sadness, suffering, impatience, anxiety, dissatisfaction, fear, surprise, and emptiness.

In the above, the emotion is preferably estimated by inference using a neural network.

In the above, the second data preferably includes the distance between the user and the external device.

In the above, the third data preferably includes the first data.

In the above, the external device preferably includes any of a car and a building.

Another embodiment of the present invention is a data processing method including a step of detecting the face of a user; a step of extracting a feature of the face from data on the detected face; a step of estimating an emotion of the user from the feature; a step of generating first data based on the emotion; a step of generating second data based on the first data; a step of determining whether the second data is transmitted to the outside on the basis of third data included in a radio wave from the Global Positioning System; and a step of transmitting the second data to the outside or a step of not transmitting the second data to the outside depending on the determination.

In the above, a step of transmitting the second data to an external device including a data reception unit, the position of which is specified by the Global Positioning System, is preferably further included after the step of determination.

In the above, the feature preferably includes at least one of the eye shape, the eyebrow shape, the mouth shape, the gaze, and the complexion of the user.

In the above, the feature is preferably extracted by inference using a neural network.

In the above, the emotion preferably includes at least one of anger, sadness, suffering, impatience, anxiety, dissatisfaction, fear, surprise, and emptiness.

In the above, the emotion is preferably estimated by inference using a neural network.

In the above, the third data preferably includes the distance between the user and the external device.

In the above, the second data preferably includes the first data.

In the above, the external device preferably includes any of a car and a building.

### Effect of the Invention

One embodiment of the present invention can provide a device or a method for preventing abnormal operation due to human emotions. Another embodiment of the present invention can provide a device or a method that allows selecting and performing of an appropriate operation according to human emotions.

Another embodiment of the present invention can provide a novel data processing device. Another embodiment of the present invention is to provide a novel data processing method.

Note that the description of these effects does not preclude the existence of other effects. One embodiment of the present invention does not need to have all these effects. Note that effects other than these can be derived from the description of the specification, the drawings, the claims, and the like.

### BRIEF DESCRIPTION OF THE DRAWINGS

FIG. 1 is a block diagram showing a structure example of a data processing device of one embodiment of the present invention.

FIG. 2A and FIG. 2B are diagrams each illustrating a neural network used in the data processing device of one embodiment of the present invention. FIG. 2C is a diagram showing an example of output results of the neural network used in the data processing device of one embodiment of the present invention.

FIG. 3 is a flowchart showing an example of a data processing method of one embodiment of the present invention.

FIG. 4 is a block diagram showing a structure example of a data processing device of one embodiment of the present invention.

FIG. 5A to FIG. 5F are diagrams showing examples of electronic devices to which one embodiment of the present invention can be applied.

### MODE FOR CARRYING OUT THE INVENTION

Embodiments will be described below with reference to the drawings. Note that the embodiments can be implemented with many different modes, and it will be readily understood by those skilled in the art that modes and details thereof can be changed in various ways without departing from the spirit and scope thereof. Thus, the present invention should not be interpreted as being limited to the following description of the embodiments.

Note that in each drawing described in this specification, the size or the region of each component is exaggerated for clarity in some cases. Therefore, the size or the region is not limited to the illustrated scale.

Note that in this specification and the like, the ordinal numbers such as “first” and “second” are used in order to avoid confusion among components and do not limit the number.

### Embodiment 1

In one embodiment of the present invention, a user's face is detected and features of the user's face are extracted from data on the detected face. The user's emotions are estimated from the extracted features, and data based on the estimated emotions is generated. The data is received by an external device including a data reception unit. In one embodiment of the present invention, a radio wave transmitted from the Global Positioning System (GPS) is received. The radio wave includes positional data of the user and the external device. The data generated by estimation of the user's emotions is transmitted according to the positional data and received by the external device. This prevents abnormal operation or incorrect operation from being caused by the user's emotions. In addition, an appropriate operation can be selected and performed according to the user's emotions.

For example, it is assumed that the data processing device of one embodiment of the present invention is a portable data terminal device such as a mobile phone (including a smart phone) or a tablet terminal and the external device receiving data transmitted from the portable data terminal device is a car including a data reception unit. The following description is made on the case where a user carries the portable data terminal device in a car and drives the car while the portable data terminal device is set so as to always detect the user's face.

Facial data includes an extreme number of features, ranging from general data such as facial contours and complexions, to local data such as the shape and positioning of facial parts such as the eyes, the eyebrows, the nose, and the mouth, as well as how the eyes open, how the nostrils bulge, how the mouth opens (or closes), the angle of the eyebrows, wrinkles between the eyebrows, and the position of the gaze. Since human facial expressions are formed by a combination of these various features, a change in facial expressions that reflects emotions of the user in driving can be detected when the portable data terminal device continues detecting the user's face. Hence, the portable data terminal device desirably detects as many facial expressions as possible.

There is a certain degree of correlation between human emotions and facial expressions, although there are individual differences and differences in degree. For example, a person who feels intense anger may show features such as more raised corners of the eyes and eyebrows, tighter gaze, and a more flushed complexion than usual. For another example, a person who is in deep sadness may show features such as more lowered corners of the eyes and eyebrows, flicker of the gaze, downcast eyes, and a paler complexion than usual. For still another example, a person who has a feeling of great surprise may show features such as the eyes widened, the mouth wide open, and staring at a single point than usual.

Thus, when the data processing device of one embodiment of the present invention detects data on the user's face and reads the user's facial expressions from the data, namely, extracts the facial features, the user's emotions of the moment can be estimated.

Note that as described above, the correlation between human emotions and facial expressions has individual differences and differences in degree, and thus is not limited to the above. Furthermore, features other than the above are shown in some cases.

The human emotions estimated by the data processing device of one embodiment of the present invention are not limited to anger, sadness, and surprise that are shown above as examples. The emotions estimated by the data processing device of one embodiment of the present invention include suffering, impatience, anxiety, dissatisfaction, fear, emptiness, and the like as well as anger, sadness, and surprise.

For example, in the case where a user feels intense anger in driving a car, the portable data terminal device installed in the car extracts, from data on the detected user's face, features such as raised corners of the eyes and eyebrows, tight gaze, and flushed complexion. These features make it possible to estimate that the user feels intense anger. As described later, the extraction of the features of the user's face and the estimation of the user's emotions can be performed by inference using a neural network.

If the estimated user's emotion is intense anger, the user's continued driving entails the risk of errors in driving operation and accidents. Therefore, the user preferably temporarily stops driving in that case.

According to one embodiment of the present invention, in the case where the portable data terminal device installed in the car estimates, for example, that the user feels anger as described above, data including an appropriate operation (e.g., slowing down the car or temporarily stopping the car) to be taken by the user is generated and transmitted to the data reception unit of the car. When the data reception unit receives the data, for example, the speed of the car is controlled so as not to exceed a certain level if the car is in motion, the engine is prevented from starting if the user has just boarded the car, and other measures are taken. This prevents abnormal operation even if the user has unusual emotions. In addition, an appropriate operation can be selected and performed according to the user's emotions.

If the user makes a mistake in driving the car, for example, if the user accidentally starts the car backward while trying to move it forward, the portable data terminal device installed in the car extracts features from data on the detected user's face, such as eyes wide open, mouth wide open, or staring at a single point. These features make it possible to estimate that the user has a feeling of surprise.

According to one embodiment of the present invention, in the case where the portable data terminal device installed in the car estimates, for example, that the user has a feeling of surprise as described above, data including an appropriate operation (e.g., immediately stopping the car) to be taken by the user is generated and transmitted to the data reception unit of the car. When the data reception unit receives the data, for example, the car is automatically stopped, and other measures are taken. This prevents abnormal operation even if the user has a sudden change in emotions. In addition, an appropriate operation can be selected and performed according to the user's emotions.

The data processing device of one embodiment of the present invention receives a radio wave transmitted from the Global Positioning System. In the case of the above example, the radio wave includes positional data of the car and the portable data terminal device (i.e., data indicating the distance between the user and the car, or the like), which is specified by the Global Positioning System. In one embodiment of the present invention, data generated by estimation of the user's emotions (e.g., stopping the car or preventing engine from starting) is transmitted to the data reception unit of the car by the portable data terminal device according to the positional data.

For example, in the case where the positional data included in the radio wave transmitted from the Global Positioning System indicates that the user is in the car where the portable data terminal device is installed, it is presumed that the user is about to drive or is in the process of driving. In that case, depending on the user's emotions, it might be unsafe to start or continue driving; thus, the data that the portable data terminal device generates by estimation of the user's emotions is transmitted to the data reception unit of the car. The car is then controlled according to the data (e.g., the engine is not started, or the speed of the car is controlled so as not to exceed a certain level).

Meanwhile, in the case where the user stays indoors such as at home while carrying the portable data terminal device, the user will not cause an accident by driving the car including the data reception unit. Thus, the data that the portable data terminal device generates by estimation of the user's emotions is not transmitted to the data reception unit of the car when the positional data included in the radio wave transmitted from the Global Positioning System indicates that there is a certain distance or longer between the user and the car including the data reception unit.

As described above, the data processing device of one embodiment of the present invention estimates user's emotions from his or her facial expressions and creates data based on the estimated emotions. Then, the data is transmitted to an external device including a data reception unit, the position of which is specified by the Global Positioning System. Note that the data is not transmitted in the case where the Global Positioning System indicates that there is a certain distance or longer between the user and the external device. Upon receiving the data, the external device executes operation based on the data.

The data processing device of one embodiment of the present invention can thus prevent abnormal operation caused by the user's emotions by operating in connection with the Global Positioning System and the external device including the data reception unit. The data processing device of one embodiment of the present invention can also select and perform an appropriate operation according to the user's emotions. For example, in the case where the data processing device of one embodiment of the present invention is a portable data terminal device and the external device including the data reception unit is a car driven by a user, the user can continue driving the car safely (e.g., driving at appropriate speed or temporarily stopping the car) even if the user has unusual emotions (e.g., anger, sadness, or surprise).

Note that the external device including the data reception unit is not limited to the car, which is given above as an example. In one embodiment of the present invention, the external device including the data reception unit may be a building. Specific examples of the building include stores in commercial facilities such as convenience stores, supermarkets, and department stores; public buildings such as banks, schools, and hospitals; residential buildings such as houses, apartments, and condominiums; and office buildings such as buildings.

More specific examples of one embodiment of the present invention will be described below with reference to drawings and a flowchart.

### Structure Example of Data Processing Device

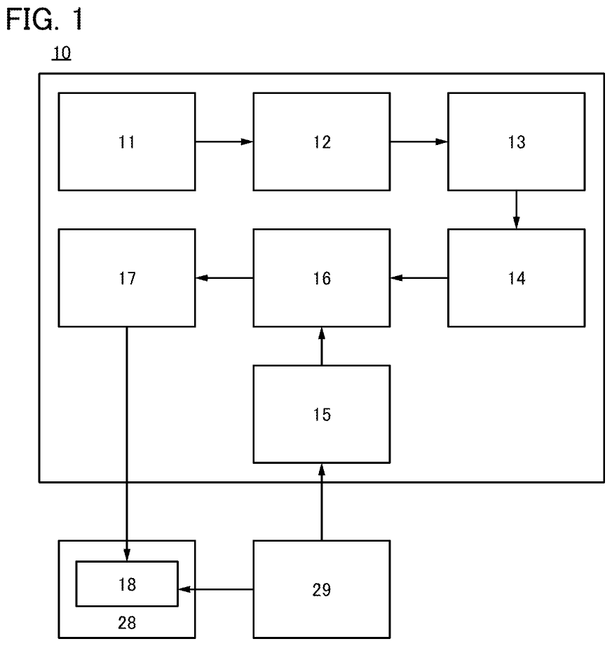

FIG. 1 is a block diagram showing a structure example of a data processing device 10 of one embodiment of the present invention. The data processing device 10 includes, as an example, a subject detection unit 11, a feature extraction unit 12, an emotion estimation unit 13, a data generation unit 14, a sensor unit 15, a data processing unit 16, and a data transmission unit 17.

As described above, the data processing device 10 can operate in connection with an external device 28 including a data reception unit 18 and a Global Positioning System 29.

Note that in the drawings attached to this specification, the block diagram is shown in which components are classified according to their functions and shown as independent blocks; however, it is difficult to separate actual components completely according to their functions, and one component may be related to a plurality of functions or a plurality of components may achieve one function.

### [Subject Detection Unit 11]

The subject detection unit 11 has a function of obtaining data on part or the whole of the user's face and outputting the data to the feature extraction unit 12.

As the subject detection unit 11, an imaging device including an image sensor can be typically used. In that case, an infrared imaging device that captures an image by irradiating the user's face with infrared rays may be used. Note that the subject detection unit 11 is not limited to an imaging device as long as a device can detect the state of part or the whole of the subject's face. An optical distance measurement device that measures the distance between the device and part of the face with the use of infrared rays or the like can also be used. A detection device that makes an electrode contact with the user's face to electrically detect muscle movement of the user's face may be used.

### [Feature Extraction Unit 12]

The feature extraction unit 12 has a function of extracting feature points from the facial data output from the subject detection unit 11, extracting features of part or the whole of the face from the position of the feature points, and outputting data on the extracted features to the emotion estimation unit 13.

When facial data obtained by the subject detection unit 11 is data on the eye and its vicinity, examples of the features that the feature extraction unit 12 extracts include a pupil, an iris, a cornea, a conjunctiva (the white of the eye), an inner canthus, an outer canthus, an upper eyelid, a lower eyelid, eyelashes, an eyebrow, a glabella, an inner end of an eyebrow, and an outer end of an eyebrow. Examples of the features other than the eye and its vicinity include a nasal root, a nasal apex, a nasal bridge, a nostril, lips (an upper lip and a lower lip), a corner of the mouth, an oral aperture, teeth, a cheek, a chin, a jaw, and a forehead. The feature extraction unit 12 recognizes the shape, position, and the like of these facial parts and extracts the position coordinates of the feature point of each part. Then, data on the extracted position coordinates or the like can be output to the emotion estimation unit 13 as data on the facial features.

In one embodiment of the present invention, the feature extraction unit 12 preferably extracts at least one of the features including the eye shape, the eyebrow shape, the mouth shape, the gaze, and the complexion from the facial data obtained by the subject detection unit 11.

As a method for extracting features by the feature extraction unit 12, a variety of algorithms for extracting a feature point from an image or the like obtained by the subject detection unit 11 can be employed. For example, an algorithm such as SIFT (Scale Invariant Feature Transform), SURF (Speeded Up Robust Features), or HOG (Histograms of Oriented Gradients) can be used.

In one embodiment of the present invention, feature extraction by the feature extraction unit 12 is preferably performed by neural network inference. It is particularly preferable to use convolutional neural networks (CNN). The case of using a neural network will be described below.

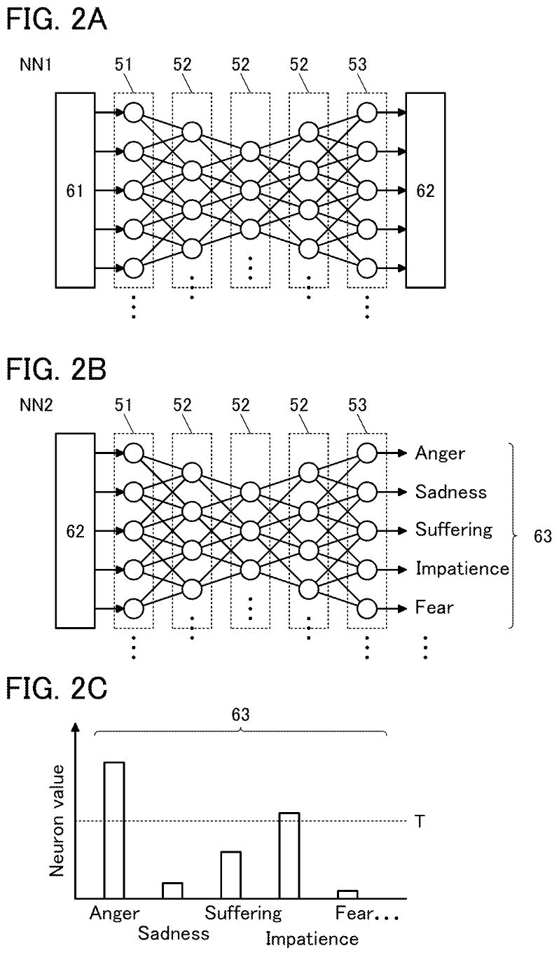

FIG. 2A schematically shows a neural network NN1 that can be used in the feature extraction unit 12. The neural network NN1 includes an input layer 51, three intermediate layers 52, and an output layer 53. Note that the number of intermediate layers 52 is not limited to three and can be one or more.

Data 61 input from the subject detection unit 11 is input to the neural network NN1. The data 61 is data that includes coordinates and a value corresponding to the coordinates. The data 61 can be typically image data that includes coordinates and a gray level corresponding to the coordinates. Data 62 is output from the neural network NN1. The data 62 is data that includes the position coordinates of the aforementioned feature point.

The neural network NN1 has learned in advance so as to extract the aforementioned feature point from the data 61 such as image data and output its coordinates. The neural network NN1 has learned such that edge computing using various filters or the like in the intermediate layers 52 increases a neuron value of the output layer 53 corresponding to the coordinates of the aforementioned feature point.

### [Emotion Estimation Unit 13]

The emotion estimation unit 13 has a function of estimating the user's emotions from data on the features of the face input from the feature extraction unit 12 and outputting data on the estimated emotions to the data generation unit 14.

The emotion estimation unit 13 can estimate whether or not the user feels negative emotions (e.g., anger, sadness, suffering, impatience, fear, anxiety, dissatisfaction, surprise, irritation, indignation, excitement, and emptiness) with the use of the data on the features of the user's face. In the case where the user feels a negative emotion, the degree (level) thereof is preferably estimated.

In one embodiment of the present invention, at least one of anger, sadness, suffering, impatience, anxiety, dissatisfaction, fear, surprise, and emptiness is preferably estimated by the emotion estimation unit 13.

In one embodiment of the present invention, emotion estimation in the emotion estimation unit 13 is preferably performed by neural network inference. It is particularly preferable to use a CNN.

FIG. 2B schematically shows a neural network NN2 that can be used in the emotion estimation unit 13. Shown here is an example where the neural network NN2 has substantially the same structure as the neural network NN1. Note that the number of neurons of the input layer 51 in the neural network NN2 can be smaller than that in the neural network NN1.

The data 62 input from the feature extraction unit 12 is input to the neural network NN2. The data 62 includes data on the coordinates of the extracted feature point.

As data input to the neural network NN2, data obtained by processing the data 62 may be used. For example, data obtained by performing calculation of a vector connecting given two feature points on all of the feature points or some of the feature points may be used as data input to the neural network NN2. Moreover, data obtained by normalizing the calculated vectors may be used. Note that hereinafter, data obtained by processing the data 62 output from the neural network NN1 is also referred to as the data 62.

Data 63 is output from the neural network NN2 to which the data 62 is input. The data 63 corresponds to neuron values output from respective neurons of the output layer 53. Each neuron of the output layer 53 is associated with one emotion. As shown in FIG. 2B, the data 63 is data that includes neuron values of the neurons each corresponding to a predetermined negative emotion (anger, sadness, suffering, impatience, fear, and the like).

The neural network NN2 has learned in advance so as to estimate the degree of the negative emotion from the data 62 and output the estimation as a neuron value. The facial expression of the user can be determined on the basis of the relative positional relationship between a plurality of feature points on the user's face. Thus, the user's emotion can be estimated from the facial expression by the neural network NN2.

FIG. 2C is a diagram schematically showing data 63. The level of a neuron value corresponding to each emotion indicates the degree of an estimated emotion. A threshold value T is indicated by a dashed line in the data 63. For example, when a neuron value corresponding to each emotion is below the threshold value T, it can be determined that the user does not feel the corresponding emotion or the degree of the corresponding emotion is low. When a neuron value corresponding to each emotion exceeds the threshold value T, the degree of the corresponding emotion can be determined to be high.

For example, it can be estimated from FIG. 2C that the user feels an emotion in which “anger” and “impatience” are mixed; in particular, the user strongly feels “anger”.

Although only one threshold value is set to determine the degree of each emotion in FIG. 2C, a plurality of threshold values may be set in accordance with the levels of the neuron values. For example, any neuron value below the threshold value T may be set as a threshold value T1 and any neuron value above the threshold value T may be set as a threshold value T2. Accordingly, the degrees of emotions can be classified into more detailed levels; for example, if the level of the neuron value corresponding to each emotion is below the threshold value T1, “the degree of emotion is low (calm)”, if it is between the threshold value T1 and the threshold value T2, “the degree emotion is somewhat high”, and if it is above the threshold value T2, “the degree of emotion is very high”.

As described above, the emotion estimation unit 13 estimates only the negative emotions and outputs the results to the data generation unit 14, so that the scale of the arithmetic operation in the emotion estimation unit 13 can be reduced, resulting in a reduction in power consumed in the arithmetic operation. In addition, the amount of data used in the data generation unit 14 can be reduced; thus, power consumed in the data transmission from the emotion estimation unit 13 to the data generation unit 14 and the arithmetic operation in the data generation unit 14 can also be reduced. Note that the emotion estimation unit 13 can estimate not only the negative emotions but also emotions opposite thereto, such as joy, appreciation, happiness, familiarity, satisfaction, and affection, and can output the results to the data generation unit 14.

Note that the emotion estimation can also be performed without using a neural network. For example, estimation may be performed by a template matching method or a pattern matching method, where an image of part of the user's face, which is obtained by the subject detection unit 11, is compared with a template image to use the degree of similarity therebetween. In that case, a structure without the feature extraction unit 12 can also be employed.

### [Data Generation Unit 14]

The data generation unit 14 has a function of determining or generating data (first data) based on the emotion estimated by the emotion estimation unit 13 and outputting the data to the data processing unit 16.

The first data is a base for data that is finally transmitted to the external device 28 including the data reception unit 18 through the data processing unit 16 and the data transmission unit 17 described later. For example, the case where the external device 28 is a car driven by a user is considered. If the emotion estimation unit 13 estimates that the user has a negative emotion exceeding the aforementioned threshold value T, the data generation unit 14 receiving the estimation result determines or generates as the first data an appropriate operation based on the user's emotion, such as “limiting the car's speed to a certain level”, “slowing down the car”, or “stopping the car”, and then outputs the first data to the data processing unit 16. If the emotion estimation unit 13 estimates that the user's negative emotion is below the aforementioned threshold value T, the data generation unit 14 receiving the estimation result determines or generates as the first data an operation to be done by the user, such as “continuing driving”, and then outputs the first data to the data processing unit 16.

Note that the first data may be linked in advance to an emotion estimated by the emotion estimation unit 13. For example, data that links the emotion “anger” to the operation “limiting the car's speed to a certain level” may be created in advance and recorded in the data generation unit 14. Thus, in the case where the emotion estimation unit 13 estimates the emotion “anger”, the data generation unit 14 can output as the first data the operation “limiting the car's speed to a certain level”. By thus creating a lot of pieces of data (data set) that link in advance each emotion to an appropriate operation, the data generation unit 14 can immediately output appropriate first data even if the user has a sudden change in emotions.

### [Sensor Unit 15]

The sensor unit 15 has a function of receiving a radio wave transmitted from the Global Positioning System 29 and outputting data (second data) included in the radio wave to the data processing unit 16.

The radio wave that the sensor unit 15 receives from the Global Positioning System 29 includes the positional data of the data processing device 10 and the external device 28 including the data reception unit 18 described later. The second data is data that includes, among the above positional data, at least the distance between the user and the external device 28. The sensor unit 15 extracts the second data from the above positional data included in the radio wave transmitted from the Global Positioning System 29 and outputs the second data to the data processing unit 16.

### [Data Processing Unit 16]

The data processing unit 16 has a function of receiving the first data output from the data generation unit 14 and the second data output from the sensor unit 15, determining or generating data (third data) based on the reception content, and outputting the third data to the data transmission unit 17.

The third data is data including the first data, which is the final data output from the data processing device 10 through the data transmission unit 17 described later. The data processing unit 16 determines or generates the third data on the basis of the first data input from the data generation unit 14. The third data includes all or at least part of the first data.

The data processing unit 16 determines whether the third data is output to the data transmission unit 17 on the basis of the second data input from the sensor unit 15. For example, the case where the data processing device 10 is a portable data terminal device of a user and the external device 28 is a car of the user is considered. If the user stays indoors such as at home while carrying the portable data terminal device, the data processing unit 16 receives from the sensor unit 15 the second data indicating that there is a certain distance or longer between the user and the car (that is, the user is not in the car).

In the above case, no matter what emotion the user has, he or she will not cause an accident by driving a car. Hence, the portable terminal device does not necessarily transmit the third data to the car. In that case, the data processing unit 16 makes a decision not to output the third data to the data transmission unit 17.

The data processing unit 16 thus generates or determines the third data on the basis of the first data input from the data generation unit 14 and determines whether the generated or determined third data is output to the data transmission unit 17 on the basis of the second data input from the sensor unit 15. Then, the third data is output to the data transmission unit 17 in the case where a decision to output the data is made.

Note that the data processing unit 16 may have two arithmetic units. For example, a structure may be employed in which one of the arithmetic units determines or generates the third data on the basis of the first data input from the data generation unit 14, and the other arithmetic unit determines whether the third data is output to the data transmission unit 17 on the basis of the second data input from the sensor unit 15 and outputs the third data to the data transmission unit 17 when a decision to output the third data is made.

### [Data Transmission Unit 17]

The data transmission unit 17 has a function of transmitting the third data input from the data processing unit 16 to the external device 28 including the data reception unit 18.

Upon receiving the third data from the data processing unit 16, the data transmission unit 17 transmits the third data to the external device 28 including the data reception unit 18, the position of which is specified by the Global Positioning System 29. Note that in one embodiment of the present invention, the external device 28 including the data reception unit 18 receiving the third data includes any of a car and a building.

The above is the description of the structure example of the data processing device 10 of one embodiment of the present invention. The use of this structure example offers the data processing device that can prevent abnormal operation caused by the user's emotions. It is also possible to provide the data processing device that can select and perform an appropriate operation according to the user's emotions.

### Example of Data Processing Method

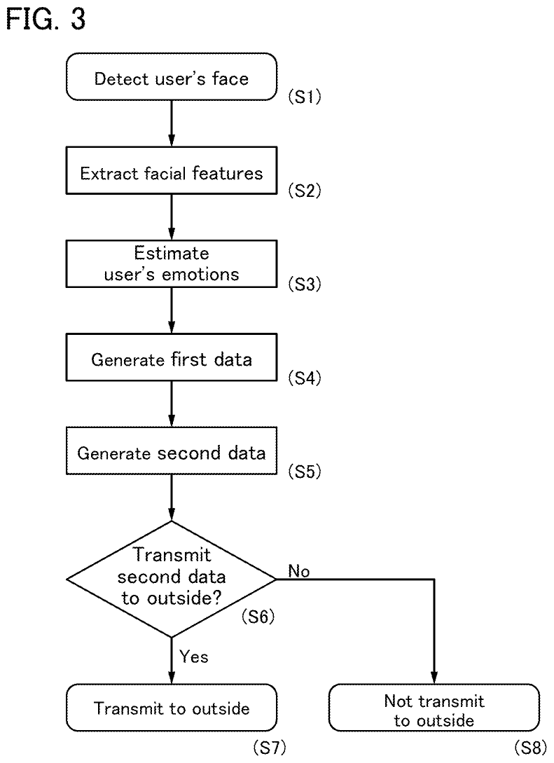

FIG. 3 is a flowchart showing an example of the data processing method of one embodiment of the present invention. A series of processes following the flowchart can be performed by the aforementioned data processing device 10 of one embodiment of the present invention.

First, part or the whole of the user's face is detected in the process of Step S1. This process can be performed by the subject detection unit 11 in the data processing device 10.

In the process of Step S2, features of part or the whole of the user's face are extracted from data on the face detected in Step S1. Note that in one embodiment of the present invention, at least one of the eye shape, the eyebrow shape, the mouth shape, the gaze, and the complexion is preferably extracted as a feature. The features are preferably extracted by inference using a neural network. This process can be performed by the feature extraction unit 12 in the data processing device 10.

In the process of Step S3, the user's emotions are estimated from the features of the user's face extracted in Step S2. In one embodiment of the present invention, at least one of anger, sadness, suffering, impatience, anxiety, dissatisfaction, fear, surprise, and emptiness is preferably estimated as a user's emotion. The emotions are preferably estimated by inference using a neural network. This process can be performed by the emotion estimation unit 13 in the data processing device 10.

In the process of Step S4, data (first data) based on the user's emotions estimated in Step S3 is determined or generated. Note that the first data here corresponds to the first data described above in <Structure example of data processing device>. This process can be performed by the data generation unit 14 in the data processing device 10.

In the process of Step S5, data (second data) based on the first data determined or generated in Step S4 is determined or generated. In one embodiment of the present invention, the second data includes all or at least part of the first data. Note that the second data here corresponds to the third data described above in <Structure example of data processing device>. This process can be performed by the data processing unit 16 in the data processing device 10.

In the process of Step S6, it is determined whether the second data determined or generated in Step S5 is transmitted to the outside or not on the basis of data (third data) included in a radio wave transmitted from the Global Positioning System. Note that the Global Positioning System here corresponds to the Global Positioning System 29 described above in <Structure example of data processing device>. The third data here corresponds to the second data described above in <Structure example of data processing device>. This process can be performed by the data processing unit 16 in the data processing device 10.

When it is determined that the second data is transmitted to the outside in Step S6, the second data is transmitted to the outside as the process based on the determination (Step S7). Note that in one embodiment of the present invention, the second data is preferably transmitted to an external device including a data reception unit, the position of which is specified by the Global Positioning System. Note that the data reception unit here corresponds to the data reception unit 18 described above in <Structure example of data processing device>. The external device here corresponds to the external device 28 described above in <Structure example of data processing device>. This process can be performed by the data transmission unit 17 in the data processing device 10.

By contrast, when it is determined that the second data is not transmitted to the outside in Step S6, the second data is not transmitted to the outside on the basis of the determination (Step S8).

In one embodiment of the present invention, the aforementioned third data is preferably data that includes the distance between the user and the external device. Also in one embodiment of the present invention, the aforementioned external device preferably includes any of a car and a building.

The above is the description of the example of the data processing method of one embodiment of the present invention. The use of this processing method example offers the data processing method that can prevent abnormal operation caused by the user's emotions. It is also possible to provide the data processing method that can select and perform an appropriate operation according to the user's emotions.

At least part of this embodiment can be implemented in combination with the other embodiments described in this specification as appropriate.

### Embodiment 2

In this embodiment, a hardware structure example of the data processing device of one embodiment of the present invention will be described. As described in Embodiment 1, the data processing device of one embodiment of the present invention can be used as a portable data terminal device such as a mobile phone (including a smart phone) or a tablet terminal.

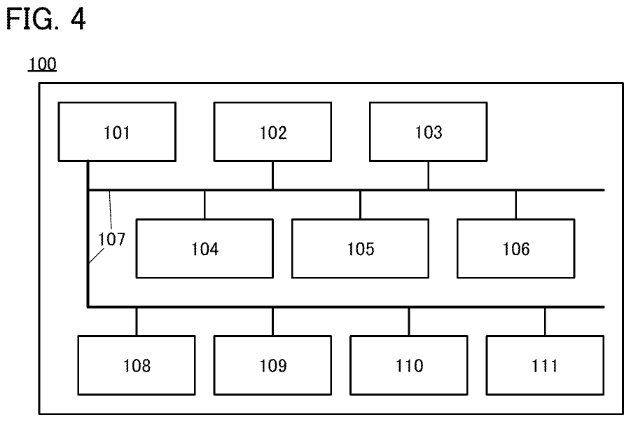

FIG. 4 shows a block diagram of a data processing device 100 described below. The data processing device 100 includes an arithmetic unit 101, an arithmetic unit 102, a memory module 103, a display module 104, a sensor module 105, a sound module 106, a communication module 108, a battery module 109, a camera module 110, an external interface 111, and the like.

The arithmetic unit 102, the memory module 103, the display module 104, the sensor module 105, the sound module 106, the communication module 108, the battery module 109, the camera module 110, the external interface 111, and the like are connected to the arithmetic unit 101 via a bus line 107.

The display module 104 can function as an image display unit of the data processing device of one embodiment of the present invention (e.g., a portable data terminal device such as a mobile phone or a tablet terminal). The sound module 106 can function as a call unit or a voice output unit of the data processing device of one embodiment of the present invention. The sensor module 105 or the camera module 110 can function as the subject detection unit 11 of the data processing device 10 described in Embodiment 1. The arithmetic unit 101, the arithmetic unit 102, and the memory module 103 can function as the feature extraction unit 12, the emotion estimation unit 13, the data generation unit 14, the data processing unit 16, and the like of the data processing device 10. The communication module 108 can function as the sensor unit 15 of the data processing device 10. The external interface 111 can function as the data transmission unit 17 of the data processing device 10.

Although the arithmetic unit 101 is denoted as one block in FIG. 4, two arithmetic units may be included. For example, in the case where the arithmetic unit 101 functions as the data processing unit 16 of the data processing device 10 described in Embodiment 1, one of the two arithmetic units can be configured to determine or generate data to be output to the data transmission unit 17 on the basis of the data input from the data generation unit 14, and the other arithmetic unit can be configured to determine whether the data determined or generated by the one arithmetic unit is output to the data transmission unit 17 on the basis of the data input from the sensor unit 15.

The arithmetic unit 101 can function as, for example, a central processing unit (CPU). The arithmetic unit 101 has a function of controlling components of the arithmetic unit 102, the memory module 103, the display module 104, the sensor module 105, the sound module 106, the communication module 108, the battery module 109, the camera module 110, the external interface 111, and the like, for example.

Signals are transmitted between the arithmetic unit 101 and the components via the bus line 107. The arithmetic unit 101 has a function of processing signals input from the components which are connected via the bus line 107, a function of generating signals to be output to the components, and the like, thereby controlling the components connected to the bus line 107 comprehensively.

The arithmetic unit 101 interprets and executes instructions from various programs with the use of a processor to process various kinds of data and control programs. Programs that might be executed by the processor may be stored in a memory region of the processor or may be stored in the memory module 103.

A CPU and other microprocessors such as a DSP (Digital Signal Processor) and a GPU (Graphics Processing Unit) can be used alone or in combination as the arithmetic unit 101. A structure may be employed in which such a microprocessor is obtained with a PLD (Programmable Logic Device) such as an FPGA (Field Programmable Gate Array) or an FPAA (Field Programmable Analog Array).

The arithmetic unit 101 may include a main memory. The main memory can have a structure in which a volatile memory such as a RAM (Random Access Memory) or a nonvolatile memory such as a ROM (Read Only Memory) is provided.

For example, a DRAM (Dynamic Random Access Memory) is used for the RAM provided in the main memory, in which case a memory space as a workspace for the arithmetic unit 101 is virtually allocated and used. An operating system, an application program, a program module, program data, and the like which are stored in the memory module 103 are loaded into the RAM to be executed. The data, program, program module, and the like which are loaded into the RAM are directly accessed and operated by the arithmetic unit 101.

Meanwhile, a BIOS (Basic Input/Output System), firmware, and the like for which rewriting is not needed can be stored in the ROM. As the ROM, a mask ROM, an OTPROM (One Time Programmable Read Only Memory), an EPROM (Erasable Programmable Read Only Memory), or the like can be used. Examples of the EPROM include a UV-EPROM (Ultra-Violet Erasable Programmable Read Only Memory) which can erase stored data by ultraviolet irradiation, an EEPROM (Electrically Erasable Programmable Read Only Memory), and a flash memory.

As the arithmetic unit 102, a processor specialized for parallel arithmetic operation as compared with a CPU is preferably used. For example, a processor including a large number of (several tens to several hundreds of) processor cores capable of parallel processing, such as a GPU, a TPU (Tensor Processing Unit), or an NPU (Neural Processing Unit), is preferably used. Accordingly, the arithmetic unit 102 can especially perform arithmetic operation by a neural network at high speed.

As the memory module 103, a memory device using a nonvolatile memory element, such as a flash memory, an MRAM (Magnetoresistive Random Access Memory), a PRAM (Phase change Random Access Memory), an ReRAM (Resistive Random Access Memory), or an FeRAM (Ferroelectric Random Access Memory); a memory device using a volatile memory element, such as a DRAM or an SRAM (Static Random Access Memory); or the like may be used, for example. Furthermore, a memory media drive such as a hard disk drive (HDD) or a solid state drive (SSD) may be used, for example.

A memory device that can be connected and disconnected through the external interface 111 with a connector, such as an HDD or an SSD, or a media drive for a recording medium such as a flash memory, a Blu-ray disc, or a DVD can be used as the memory module 103. Note that the memory module 103 is not incorporated in the data processing device 100, and a memory device located outside may be used as the memory module 103. In that case, the memory device may be connected through the external interface 111, or data transmission and reception may be wirelessly performed using the communication module 108.

The display module 104 includes a display panel, a display controller, a source driver, a gate driver, and the like. An image can be displayed on a display surface of the display panel. The display module 104 may further include a projection portion (screen) to employ a method in which an image displayed on the display surface of the display panel is projected on the screen. In that case, when a material that transmits visible light is used for the screen, an AR device in which a displayed image is superimposed on a background image can be obtained.

Examples of a display element that can be used for the display panel include a liquid crystal element, an organic EL element, an inorganic EL element, an LED element, a microcapsule, an electrophoretic element, an electrowetting element, an electrofluidic element, an electrochromic element, and a MEMS element.

A touch panel having a touch sensor function can be used as the display panel. In that case, the display module 104 includes a touch sensor controller, a sensor driver, and the like. As the touch panel, an on-cell touch panel or an in-cell touch panel in which a display panel and a touch sensor are combined is preferable. The on-cell or in-cell touch panel can be thin and lightweight. The on-cell or in-cell touch panel has fewer components and can therefore reduce cost.

The sensor module 105 includes a sensor unit and a sensor controller. The sensor controller converts the input from the sensor unit into a control signal and outputs it to the arithmetic unit 101 via the bus line 107. The sensor controller may handle errors made by the sensor unit or may calibrate the sensor unit. Note that the sensor controller may include a plurality of controllers which control the sensor unit.

The sensor unit included in the sensor module 105 preferably includes a photoelectric conversion element that detects visible light, infrared rays, ultraviolet rays, or the like and outputs the detection intensity thereof. In that case, the sensor unit can be called an image sensor unit.

The sensor module 105 preferably includes, in addition to the sensor unit, a light source emitting visible light, infrared rays, or ultraviolet rays. In particular, in the case where the sensor module 105 is used for detecting part of the user's face, including a light source emitting infrared rays enables an image to be captured with high sensitivity without making the user feel the glare.

The sensor module 105 may include a variety of sensors which have a function of measuring force, displacement, position, speed, acceleration, angular velocity, rotational frequency, distance, light, liquid, magnetism, temperature, a chemical substance, a sound, time, hardness, electric field, current, voltage, electric power, radiation, flow rate, humidity, gradient, oscillation, smell, or infrared rays.

The sound module 106 includes an audio input unit, an audio output unit, a sound controller, and the like. The audio input unit includes a microphone, an audio input connector, or the like, for example. The audio output unit includes a speaker, an audio output connector, or the like, for example. The audio input unit and the audio output unit are connected to the sound controller, and are connected to the arithmetic unit 101 via the bus line 107. Audio data input to the audio input unit is converted into a digital signal in the sound controller and then processed in the sound controller and the arithmetic unit 101. By contrast, the sound controller generates an analog audio signal audible to a user in response to instructions from the arithmetic unit 101 and outputs it to the audio output unit. To the audio output connector of the audio output unit, an audio output device such as earphones, headphones, or a headset can be connected and a sound generated in the sound controller is output to the device.

The communication module 108 can perform communication via an antenna. For example, the communication module 108 can have a function of receiving a radio wave from the Global Positioning System 29 described in Embodiment 1 and outputting data included in the radio wave to the data processing unit 16 of the data processing device 10. For another example, the communication module 108 can have a function of controlling a control signal for connecting the data processing device 100 to a computer network in response to instructions from the arithmetic unit 101 and transmitting the signal to the computer network. Accordingly, communication can be performed by connecting the data processing device 100 to a computer network such as the Internet, an intranet, an extranet, a PAN (Personal Area Network), a LAN (Local Area Network), a CAN (Campus Area Network), a MAN (Metropolitan Area Network), a WAN (Wide Area Network), or a GAN (Global Area Network). In the case where a plurality of communication methods are used, a plurality of antennas for the communication methods may be included.

The communication module 108 is provided with a high frequency circuit (RF circuit), for example, to transmit and receive an RF signal. The high frequency circuit is a circuit for performing mutual conversion between an electromagnetic signal and an electric signal in a frequency band that is set by national laws to perform wireless communication with another communication apparatus using the electromagnetic signal. As a practical frequency band, several tens of kilohertz to several tens of gigahertz are generally used. A structure can be employed in which the high frequency circuit connected to an antenna includes a high frequency circuit portion compatible with a plurality of frequency bands and the high frequency circuit portion includes an amplifier, a mixer, a filter, a DSP, an RF transceiver, or the like. In the case of performing wireless communication, it is possible to use, as a communication protocol or a communication technology, a communications standard such as LTE (Long Term Evolution), or a communications standard developed by IEEE, such as Wi-Fi (registered trademark) or Bluetooth (registered trademark).

The communication module 108 may have a function of connecting the data processing device 100 to a telephone line. The communication module 108 may include a tuner for generating a video signal, which is to be output to the display module 104, from airwaves received by the antenna.

The battery module 109 can include a secondary battery and a battery controller. Typical examples of the secondary battery include a lithium-ion secondary battery and a lithium-ion polymer secondary battery. The battery controller can have a function of supplying power accumulated in a battery to the components, a function of receiving power supplied from the outside and charging the battery, and a function of controlling the charging operation in response to the charge state of the battery, for example. The battery controller can include a BMU (Battery Management Unit), for example. The BMU collects data on cell voltage or cell temperatures of the battery, monitors overcharge and overdischarge, controls a cell balancer, handles a deterioration state of the battery, calculates the remaining battery power level (State Of Charge: SOC), and controls detection of a failure, for example.

The camera module 110 can include an imaging element and a controller. A still image or a moving image can be captured at the press of a shutter button or by the operation of the touch panel of the display module 104, for example. The captured image or video data can be stored in the memory module 103. The image or the video data can be processed in the arithmetic unit 101 or the arithmetic unit 102. The camera module 110 may include a light source for capturing images. For example, a lamp such as a xenon lamp, a light-emitting element such as an LED or an organic EL, or the like can be used. Alternatively, light emitted from the display panel of the display module 104 may be used as the light source for capturing images; in that case, light of various colors besides white may be used for capturing images.

As an external port of the external interface 111, for example, a transceiver for optical communication using infrared rays, visible light, ultraviolet rays, or the like may be provided or a transceiver of an RF signal may be provided as in the aforementioned communication module 108. Such a structure enables the external interface 111 to function as the data transmission unit 17 of the data processing device 10 described in Embodiment 1, so that data determined or generated in the data processing unit 16 can be transmitted to the external device 28 including the data reception unit 18.

In addition to the above structure, for example, a physical button may be provided on the housing of the data processing device 100 or an external port to which another input component can be connected may be provided. In that case, the external port included in the external interface 111 can be connected to a device, e.g., an input means such as a keyboard or a mouse, an output means such as a printer, or a storage means such as an HDD, through a cable. A USB terminal is a typical example. As the external port, a LAN connection terminal, a digital broadcast-receiving terminal, an AC adaptor connection terminal, or the like may be provided.

The above is the description of the hardware structure of the data processing device of one embodiment of the present invention.

At least part of this embodiment can be implemented in combination with the other embodiments described in this specification as appropriate.

### Embodiment 3

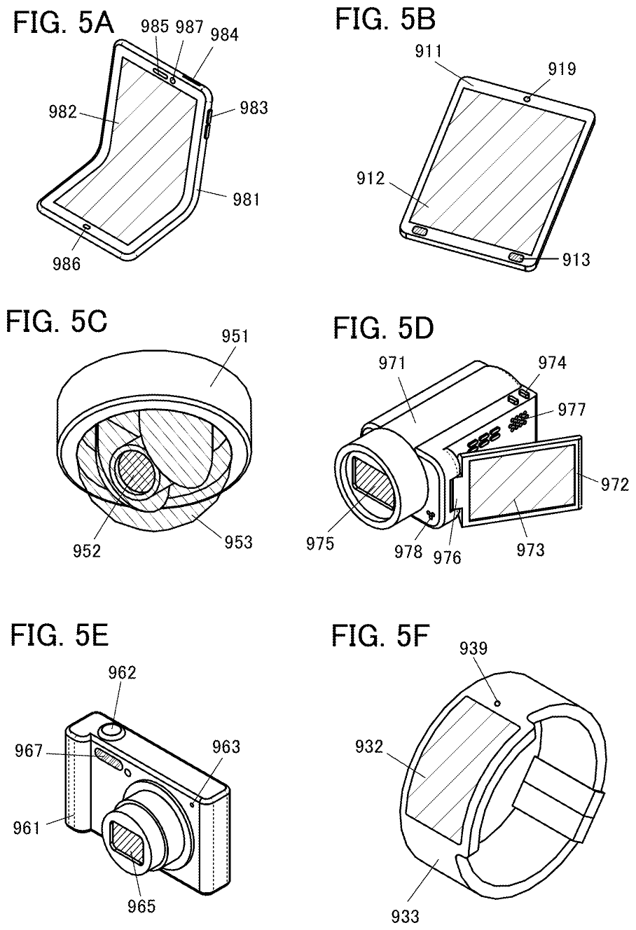

Examples of an electronic device to which one embodiment of the present invention can be applied include display devices, personal computers, image memory devices or image reproducing devices provided with storage media, mobile phones (including smart phones), game machines including portable game machines, portable data terminals (tablet terminals), e-book readers, cameras such as video cameras and digital still cameras, goggle-type displays (head mounted displays), navigation systems, audio reproducing devices (e.g., car audio players and digital audio players), copiers, facsimiles, printers, multifunction printers, automated teller machines (ATM), and vending machines. FIG. 5A to FIG. 5F show specific examples of such electronic devices.

FIG. 5A is an example of a mobile phone, which includes a housing 981, a display portion 982, an operation button 983, an external connection port 984, a speaker 985, a microphone 986, a camera 987, and the like. The display portion 982 of the mobile phone includes a touch sensor. Operations such as making a call and inputting text can be performed by touch on the display portion 982 with a finger, a stylus, or the like. The data processing device and the data processing method of one embodiment of the present invention can be used for components for acquiring images (acquiring data on the user's face) in the mobile phone.

FIG. 5B is an example of a portable data terminal, which includes a housing 911, a display portion 912, a speaker 913, a camera 919, and the like. A touch panel function of the display portion 912 enables input and output of data. Furthermore, a character or the like in an image that is captured by the camera 919 can be recognized and the character can be voice-output from the speaker 913. The data processing device and the data processing method of one embodiment of the present invention can be used for components for acquiring images (acquiring data on the user's face) in the portable data terminal.

FIG. 5C is an example of a surveillance camera (a security camera), which includes a support base 951, a camera unit 952, a protection cover 953, and the like. The camera unit 952 is provided with a rotation mechanism and the like and can capture an image of all of the surroundings when provided on a ceiling. The data processing device and the data processing method of one embodiment of the present invention can be used for components for acquiring images (acquiring data on the user's face) in the camera unit. Note that a surveillance camera is a name in common use and does not limit the use thereof. A device that has a function as a surveillance camera is also referred to as a camera or a video camera, for example.

FIG. 5D is an example of a video camera, which includes a first housing 971, a second housing 972, a display portion 973, operation keys 974, a lens 975, a connection portion 976, a speaker 977, a microphone 978, and the like. The operation keys 974 and the lens 975 are provided for the first housing 971, and the display portion 973 is provided for the second housing 972. The data processing device and the data processing method of one embodiment of the present invention can be used for components for acquiring images (acquiring data on the user's face) in the video camera.

FIG. 5E is an example of a digital camera, which includes a housing 961, a shutter button 962, a microphone 963, a light-emitting portion 967, a lens 965, and the like. The data processing device and the data processing method of one embodiment of the present invention can be used for components for acquiring images (acquiring data on the user's face) in the digital camera.

FIG. 5F is an example of a wrist-watch-type data terminal, which includes a display portion 932, a housing 933 also serving as a wristband, a camera 939, and the like. The display portion 932 is provided with a touch panel for operating the data terminal. The display portion 932 and the housing 933 also serving as a wristband have flexibility and fit a body well. The data processing device and the data processing method of one embodiment of the present invention can be used for components for acquiring images (acquiring data on the user's face) in the data terminal.

For example, the case where the data processing device of one embodiment of the present invention is the mobile phone shown in FIG. 5A is considered. For example, when the user drives a car including the data reception unit 18 described in Embodiment 1, by installing the mobile phone in a position where the user's face can be detected, the user can always drive safely regardless of his/her emotions by the data processing method of one embodiment of the present invention.

The data processing device of one embodiment of the present invention that can be used in driving is not limited to the mobile phone shown in FIG. 5A. The data processing device may be the portable data terminal shown in FIG. 5B, the video camera shown in FIG. 5D, the digital camera shown in FIG. 5E, or the wrist-watch-type data terminal shown in FIG. 5F.

For another example, the case where the data processing device of one embodiment of the present invention is the mobile phone shown in FIG. 5A and the user makes a call with the mobile phone in a building including the data reception unit 18 described in Embodiment 1 is considered. In that case, the mobile phone can detect the user's face in calling. For example, when the mobile phone estimates from the user's facial expression that the user's emotions have suddenly turned into intense anger, it transmits data to stop the call. Upon receiving the data, the building including the data reception unit 18 can take measures such as forcibly terminating the user's call (e.g., sending a signal to automatically disconnect the call). This prevents the deterioration of interpersonal relationships and loss of opportunities for business transactions.

For still another example, the case where the data processing device of one embodiment of the present invention is the mobile phone shown in FIG. 5A and the external device 28 including the data reception unit 18 described in Embodiment 1 is a building equipped with an automatic teller machine (e.g., a bank or a convenience store) is considered. It is supposed that, for example, the user notices that he or she has received an email on the mobile phone asking the user to make a transfer. At this time, the mobile phone estimates from the user's facial expression that the user has a strong feeling of anxiety. In the case where the user is close to the automatic teller machine, the data processing method of one embodiment of the present invention can take measures such as sending data from the mobile phone to stop the use of the building equipped with the automatic teller machine. This prevents the aforementioned bank transfer scams and other forms of damage.

For still another example, the case where the data processing device of one embodiment of the present invention is the surveillance camera shown in FIG. 5C is considered. It is supposed that, for example, the surveillance camera is installed at the cash register of a convenience store and the external device 28 including the data reception unit 18 described in Embodiment 1 is a crisis management office of the convenience store. For example, if a convenience store clerk is having a hard time dealing with an annoying customer, and the surveillance camera estimates from the clerk's facial expression that the clerk has a strong feeling of distress, it is possible to take measures such as sending data from the surveillance camera to the crisis management office requesting the dispatch of support personnel. This prevents the clerk from getting into trouble and other damage.

At least part of this embodiment can be implemented in combination with the other embodiments described in this specification as appropriate.

### REFERENCE NUMERALS

10: data processing device, 11: subject detection unit, 12: feature extraction unit, 13: emotion estimation unit, 14: data generation unit, 15: sensor unit, 16: data processing unit, 17: data transmission unit, 18: data reception unit, 28: external device, 29: the Global Positioning System, 51: input layer, 52: intermediate layer, 53: output layer, 61: data, 62: data, 63: data, 100: data processing device, 101: arithmetic unit, 102: arithmetic unit, 103: memory module, 104: display module, 105: sensor module, 106: sound module, 107: bus line, 108: communication module, 109: battery module, 110: camera module, 111: external interface, 911: housing, 912: display portion, 913: speaker, 919: camera, 932: display portion, 933: housing also serving as a wristband, 939: camera, 951: support base, 952: camera unit, 953: protection cover, 961: housing, 962: shutter button, 963: microphone, 965: lens, 967: light-emitting portion, 971: first housing, 972: second housing, 973: display portion, 974: operation key, 975: lens, 976: connection portion, 977: speaker, 978: microphone, 981: housing, 982: display portion, 983: operation button, 984: external connection port, 985: speaker, 986: microphone, 987: camera

1. A data processing device comprising:
a subject detection unit detecting a face of a user;
a feature extraction unit extracting a feature of the face;
an emotion estimation unit estimating an emotion of the user from the feature;
a data generation unit generating first data based on the estimated emotion;
a sensor unit receiving a radio wave from the Global Positioning System;
a data processing unit receiving the first data and second data included in the radio wave and transmitted from the sensor unit and generating third data based on the first data and the second data; and
a data transmission unit transmitting the third data.

2. The data processing device according to claim 1,
wherein the third data is transmitted to an external device including a data reception unit, the position of the external device being specified by the Global Positioning System.

3. The data processing device according to claim 1,
wherein the feature includes at least one of an eye shape, an eyebrow shape, a mouth shape, gaze, and a complexion of the user.

4. The data processing device according to claim 1,
wherein the feature is extracted by inference using a neural network.

5. The data processing device according to claim 1,
wherein the emotion includes at least one of anger, sadness, suffering, impatience, anxiety, dissatisfaction, fear, surprise, and emptiness.

6. The data processing device according to claim 1,
wherein the emotion is estimated by inference using a neural network.

7. The data processing device according to claim 1,
wherein the second data includes a distance between the user and the external device.

8. The data processing device according to claim 1,
wherein the third data includes the first data.

9. The data processing device according to claim 1,
wherein the external device includes any of a car and a building.

10. A data processing method comprising:
a step of detecting a face of a user;
a step of extracting a feature of the face from data on the detected face;
a step of estimating an emotion of the user from the feature;
a step of generating first data based on the emotion;
a step of generating second data based on the first data;
a step of determining whether the second data is transmitted to outside on the basis of third data included in a radio wave from the Global Positioning System; and
a step of transmitting the second data to the outside or a step of not transmitting the second data to the outside depending on the determination.

11. The data processing method according to claim 10, further comprising:
after the step of determination, a step of transmitting the second data to an external device including a data reception unit, the position of the external device being specified by the Global Positioning System.

12. The data processing method according to claim 10,
wherein the feature includes at least one of an eye shape, an eyebrow shape, a mouth shape, gaze, and a complexion of the user.

13. The data processing method according to claim 10,
wherein the feature is extracted by inference using a neural network.

14. The data processing method according to claim 10,
wherein the emotion includes at least one of anger, sadness, suffering, impatience, anxiety, dissatisfaction, fear, surprise, and emptiness.

15. The data processing method according to claim 10,
wherein the emotion is estimated by inference using a neural network.

16. The data processing method according to claim 10,
wherein the third data includes a distance between the user and the external device.

17. The data processing method according to claim 10,
wherein the second data includes the first data.

18. The data processing method according to claim 10,
wherein the external device includes any of a car and a building.
